SEQUENCE LISTING (7 sequences)

SEQ ID NO: 1 - protein, 4 aa, Artificial Sequence
  source: /note=“Description of Artificial Sequence Synthetic peptide”
RADA

SEQ ID NO: 2 - protein, 4 aa, Artificial Sequence
  source: /note=“Description of Artificial Sequence Synthetic peptide”
KLDL

SEQ ID NO: 3 - protein, 4 aa, Artificial Sequence
  source: /note=“Description of Artificial Sequence Synthetic peptide”
IEIK

SEQ ID NO: 4 - protein, 200 aa, Artificial Sequence
  source: /note=“Description of Artificial Sequence Synthetic polypeptide”
  MISC_FEATURE (1)..(200): /note=“This sequence may encompass 2-50 ′Arg Ala Asp Ala′ repeating units”
RADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADARADA

SEQ ID NO: 5 - protein, 12 aa, Artificial Sequence
  source: /note=“Description of Artificial Sequence Synthetic peptide”
KLDLKLDLKLDL

SEQ ID NO: 6 - protein, 13 aa, Artificial Sequence
  source: /note=“Description of Artificial Sequence Synthetic peptide”
IEIKIEIKIEIKI

SEQ ID NO: 7 - protein, 16 aa, Artificial Sequence
  source: /note=“Description of Artificial Sequence Synthetic peptide”
RADARADARADARADA

(19) United States
(12) Patent Application Publication
(10) Pub. No.: US 2019/0358290 A1
(43) Pub. Date: Nov. 28, 2019

(54) Self Assembling Peptide Matrix for the Prevention of Esophageal Stricture After Endoscopic Dissection

(71) Applicants: 3-D Matrix, Ltd., Tokyo (JP); ASSISTANCE PUBLIQUE-HOPITAUX DE PARIS (AP-HP), Paris (FR)

(72) Inventors: Eun Seok Gil, Lexington, MA (US); Marie Reveiller-Buffier, Sathonay-Camp (FR); Frederic Prat, Sceaux (FR); Maximilien Louis Francois Benjamin Barret, Paris (FR)

(21) Appl. No.: 16/312,878

(22) PCT Filed: Jun. 27, 2017
(86) PCT No.: PCT/US2017/039407

§ 371 Date: Dec. 21, 2018

Related U.S. Application Data
(60) Provisional application No. 62/355,111, filed on Jun. 27, 2016.

(51) Int. Cl. A61K 38/10; A61K 31/573; A61K 9/70; A61K 9/08; A61K 38/18; A61K 9/00; A61B 1/267

(52) U.S. Cl. CPC A61K 38/10; A61K 31/573; A61K 9/70; A61B 1/267; A61K 38/1866; A61K 9/0053; A61K 9/08

(57) ABSTRACT

Methods for preventing esophageal stricture following an endoscopic resection procedure in a subject are provided. A solution having a pH level of about 3.5 and including a self-assembling peptide comprising between about 7 amino acids and about 32 amino acids in an effective amount and in an effective concentration to form a hydrogel under esophageal conditions to provide prevention of esophageal stricture may be introduced to a target site.

### FIELD OF THE DISCLOSURE

This disclosure generally relates to methods that may be used in medical and research applications. More particularly, this disclosure relates to methods that may be used to prevent esophageal stricture after endoscopic dissection.

### BACKGROUND

Endoscopic dissection has become the cornerstone of the diagnostic and therapeutic management of preneoplastic states and early esophageal neoplasia in the esophagus. However, mucosal defects exceeding three quarters of the esophageal circumference (270°) induce the formation of an esophageal stricture in about 90% of the cases. Theses strictures may require numerous endoscopic dilation procedures with significant patient discomfort and risk of esophageal perforation.

### SUMMARY

In accordance with one or more aspects, a method of preventing esophageal stricture following an endoscopic resection procedure in a subject may comprise introducing a delivery device to an esophagus of the subject, positioning an end of the delivery device at a target area of the esophagus where prevention of esophageal stricture is desired, administering through the delivery device a solution having a pH level of about 3.5 or less, the solution including a self-assembling peptide comprising between about 7 amino acids and about 32 amino acids in an effective amount and in an effective concentration to form a hydrogel under esophageal conditions to provide prevention of esophageal stricture, and removing the delivery device from the esophagus.

In some aspects, the endoscopic resection procedure relates to superficial neoplasm of the esophagus. The endoscopic resection procedure may be either circumferential endoscopic submucosal dissection or circumferential endoscopic mucosal resection. Administering the solution may provide prevention of inflammatory and/or fibrotic patterns. Administering the solution may speed reepithelialization and/or inhibit acid aggression at the target area of the esophagus. Administering the solution may provide a scaffold for epithelial cell migration and/or healing at the target area of the esophagus.

In some aspects, the target area of the esophagus is located about 5 cm above an esophagogastric junction. At least one of the effective amount and the effective concentration may be based in part on a dimension of the target area of the esophagus. In some non-limiting aspects, the target area of the esophagus may be between about 1 cm and about 10 cm in dimension. For example, the target area of the esophagus may be about 5 cm in dimension. The effective amount may be approximately 1 mL per 1 cm² of target area. The concentration effective to provide prevention of esophageal stricture may comprise a concentration in a range of about 0.1 weight per volume (w/v) percent to about 3 w/v percent peptide. The amount effective to provide prevention of esophageal stricture may comprise a non-limiting volume in a range of about 0.1 mL to about 10 mL.

In some aspects, the method may further comprise administering a corticosteroid at the target area of the esophagus. The method may further comprise visualizing a region comprising at least a portion of the esophagus. The method may further comprise monitoring the target area of the esophagus to determine an effectiveness of the administration of the solution. The target area may be monitored via endoscopy and/or radiology. The target area may be monitored at days 3, 7, 14, 21, and 28 following administration of the solution.

In some aspects, the solution is substantially free of cells. The solution may be substantially free of drugs. The solution may consist essentially of an amphiphilic peptide comprising at least 12 amino acids that alternate between a hydrophobic amino acid and a hydrophilic amino acid. The solution may consist of an amphiphilic peptide comprising at least 12 amino acids that alternate between a hydrophobic amino acid and a hydrophilic amino acid. In some aspects, the subject may be a mammal. In some non-limiting aspects, the subject may be human. The method may further comprise evaluating the subject to determine a need for preventing esophageal stricture and preparing the solution. The method may further comprise introducing an endoscope into the esophagus prior to introducing the delivery device. In some aspects, the solution may further comprise at least one biologically active agent. The peptide in the solution may comprise RADA16. In other embodiments, the peptide in the solution may comprise IEIK13.

In some aspects, administration of the solution may maintain a predetermined lumen dimension at the target area of the esophagus. The predetermined lumen dimension may be at least about 10 mm. The solution may be buffered with an alkali salt. The solution may be buffered with sodium hydroxide, sodium chloride, potassium hydroxide, calcium hydroxide, sodium carbonate, sodium acetate, or sodium sulfide. In some aspects, the pH level of the solution is about 3.4. The solution may be characterized by a storage modulus of between about 100 Pa-1000 Pa, at 2.5% concentration when measured at 1 rad/sec and 1 Pa of stress. The solution may further comprise one or more isotonic agents including salts, sugars, and mixtures thereof to control the tonicity, and one or more alkali salts or acidic salts to control the pH level. The solution may further comprise an anti-inflammatory molecule and/or a wound healing stimulant. In some embodiments, administering the solution may comprise administering the solution in at least two doses.

### BRIEF DESCRIPTION OF THE DRAWINGS

The accompanying drawings are not intended to be drawn to scale. For purposes of clarity, not every component may be labeled. In the drawings:

FIGS. 1-3 present data discussed in the accompanying Example.

### DETAILED DESCRIPTION

Methods of the present disclosure may prevent esophageal stricture after endoscopic dissection.

Endoscopic mucosal resection (EMR) and endoscopic submucosal dissection (ESD) are primary surgical options for resection of lesion sites such as polyps and cancerous tumors in a digestive system or gastrointestinal tract. EMR and ESD are both minimally invasive surgeries. ESD can generally dissect wider areas of a lesion site than EMR. ESD therefore may cause obstructions due to scar contraction/shrinking during the healing process more often than EMR. However, both EMR and ESD procedures may cause lesions that induce post-surgical gastrointestinal obstruction during the course of healing. One form of gastrointestinal obstruction may be a stenosis in the gastrointestinal tract during the course of healing. By stenosis it is meant a narrowing in a tubular organ or structure, such as the gastrointestinal tract, which may lead to a partial or full obstruction in the gastrointestinal tract. Subjects may typically need to undergo several treatments of balloon dilatation and local steroid injection leading to a significant drop in quality of life.

Extensive mucosal resection for superficial neoplasm of the esophagus, either by circumferential EMR or circumferential ESD, may result in a very high rate of esophageal stricture or stenosis, for example, at least about 80%. This presents a major limitation of endoscopic therapy. Without wishing to be bound by any particular theory, stricture is thought to involve luminal stress factors: mechanical factors resulting from food boluses and esophageal peristaltism, microbiological factors originating from the esophageal and oro-pharyngeal microbiome, and chemical factors resulting from gastric refluxate. The exposure of the submucosal space to these factors may induce an inflammatory and fibrogenic reaction leading to esophageal stricture. These strictures exhibit inflammatory and fibrotic patterns which are likely to be explained by slow reepithelialization and acid aggression of the esophageal wound. Besides the administration or corticosteroids, which results in possible major side effects, no treatment has yet proven effective to prevent esophageal stricture.

Several strategies for post endoscopic stricture prevention have been proposed. Anti-inflammatory, antifibrotic, or antimitotic drugs have been administered in order to suppress fibrogenesis in the esophageal wall. Systemic administration of corticosteroids is another option, however with a suboptimal efficacy, and a risk of severe infectious morbidity. Yet another option is to protect the esophageal wound from luminal stress, either by stimulating the epithelial growth with growth factor, epithelial cell sheets, or by covering the wound with mineral or organic wound dressings.

“Preventing” may include complete prevention in that a target site may return to a pre-operative state or other “normal” state. Preventing may include at least partially preventing or at least partially reducing, which may include returning the target site to a state that allows at least some relief or that returns the target site to a lesser than normal state.

The methods may comprise preventing or reducing esophageal stricture after endoscopic resection in a subject. As used herein, the term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

The prevention or reduction of esophageal stricture may be partial or complete. The methods may include administration, application, or injection of a self-assembling peptide, or a solution comprising a self-assembling peptide, or a composition comprising a self-assembling peptide, to a predetermined or desired target area. The self-assembling peptide may serve as a wound covering agent, providing both protection of the wound from acid aggression, and a scaffold for epithelial cell migration and wound healing.

The term “self-assembling peptide” may refer to a peptide that may exhibit a beta-sheet structure in aqueous solution in the presence of specific conditions to induce the beta-sheet structure. These specific conditions may include increasing the pH of a self-assembling peptide solution. The increase in pH may be an increase in pH to a physiological pH. The specific conditions may include conditions related to a gastrointestinal tract, such as an esophagus.

In some non-limiting embodiments, peptide hydrogels such as those disclosed in International Patent Application Publication No. WO2015/138514 titled “SELF-ASSEMBLING PEPTIDE COMPOSITIONS” and assigned to 3-D Matrix, Ltd., which is hereby incorporated herein by reference in its entirety for all purposes, may be implemented.

Embodiments disclosed herein may comprise certain peptide compositions (and particularly certain compositions of self-assembling peptide agents), and technologies relating thereto. In some embodiments, such compositions may be or comprise solutions. In some embodiments, such compositions may be or comprise gels. In some embodiments, such compositions may be or comprise solid (e.g., dried/lyophilized) peptides. For example, particular peptide compositions (i.e., peptide compositions having specific concentration, ionic strength, pH, viscosity and/or other characteristics) have useful and/or surprising attributes (e.g., gelation or self-assembly kinetics [e.g., rate of gelation and/or rate and reversibility of peptide self-assembly], stiffness [e.g., as assessed via storage modulus], and/or other mechanical properties).

In some embodiments, peptides included in provided compositions are self-assembling peptides. In some embodiments, peptides included in provided compositions are amphiphilic peptides. In some embodiments, peptides included in provided compositions have an amino acid sequence characterized by at least one stretch (e.g., of at least 4, 5, 6, 7, 8, 9, 10, 11, 12, 13, 14, 15, 16, 17, 18, 19, 20 etc amino acids) of alternating hydrophilic and hydrophobic amino acids. In accordance with one or more embodiments, peptide compositions may include an amphiphilic polypeptide having about 6 to about 200 amino acid residues. In some embodiments, a peptide may have a length within the range of about 6 to about 20 amino acids and an amino acid sequence of alternating hydrophobic amino acid and hydrophilic amino acids.

In some embodiments, peptides included in provided compositions have an amino acid sequence that includes one or more repeats of Arg-Ala-Asp-Ala (RADA). In some embodiments, peptides included in provided compositions have an amino acid sequence that comprises or consists of repeated units of the sequence Lys-Leu-Asp-Leu (KLDL). In some embodiments, peptides included in provided compositions have an amino acid sequence that comprises or consists of repeated units of the sequence Ile-Glu-Ile-Lys (IEIK). In some embodiments, the peptides may be IEIK13, KLD12, or RADA16. In some embodiments, compositions of these peptides may have enhanced properties relative to appropriate reference compositions that have different (e.g., lower) pH level, and/or ionic strength.

In some embodiments, increased ionic strength may beneficially impact stiffness and/or gelation kinetics to peptide compositions rendering them suitable for a broader range of applications. In some embodiments, increased ionic strength may be physiological ionic strength, which may occur when peptide compositions are placed into the body. In some embodiments, an ionic strength of a peptide composition may be about 0.0001 M to about 1.5 M. In some embodiments, an ionic strength of a peptide composition may be adjusted by mixing common salts, for example, NaCl, KCl, MgCl₂, CaCl₂, CaSO₄, DPBS (Dulbecco's Phosphate-Buffered Saline, 10×). In some embodiments, ionic strengths of peptide compositions may be adjusted by mixing common salts, wherein one or more common salts are composed of one or more salt forming cations and one or more salt forming anions, wherein the salt forming cations are selected from the group consisting of ammonium, calcium, iron, magnesium, potassium, pyridinium, quaternary ammonium, and sodium, wherein the salt forming anions are selected from the group consisting of acetate, carbonate, chloride, citrate, cyanide, floride, nitrate, nitrite, and phosphate.

In some embodiments, a peptide composition may be solution, gel, or any combination thereof. In some embodiments, peptide concentration in a peptide composition is at least 0.05%, at least 0.25%, at least 0.5%, at least 0.75%, at least 1.0% or more. In some embodiments, peptide concentration in a peptide composition is less than 5%, less than 4.5%, less than 4%, less than 3.5%, less than 3%, or less. In some embodiments, peptide concentration in a peptide composition is within a range between about 0.5% and about 3%. In some embodiments, peptide concentration in a peptide composition is within a range between about 0.5% and about 2.5%. In some embodiments, peptide concentration in a peptide composition is within a range between about 1% and about 3%. In some embodiments, peptide concentration in a peptide composition is within a range between about 1% and about 2.5%. In some embodiments, peptide concentration in a peptide composition is about 0.5%, about 1%, about 1.5%, about 2%, about 2.5%, about 3%, or more. In some particular embodiments, where the peptide is RADA16, peptide concentration in peptide compositions is within a range of about 0.05% to about 10%.

In some embodiments, a peptide composition may have a viscosity with the range of about 1 to about 10000 Pa-S. In some embodiments, a peptide composition may have a storage modulus with the range of about 50 to about 2500 Pa.

In accordance with one or more embodiments, a pH level of a peptide hydrogel may be adjusted with a buffer, such as an alkali salt buffer. Various salts, such as alkali salts including but not limited to sodium hydroxide, sodium chloride, potassium hydroxide, calcium hydroxide, sodium carbonate, sodium acetate, or sodium sulfide, may be used to buffer the hydrogel formulations. As a desired pH level is approached, addition of buffer may be carefully regulated.

In accordance with one or more embodiments, a pH level of the peptide hydrogel may impact mechanical strength and/or rate of self-assembly of the peptides. For example, the rate of self-assembly upon contact with physiological medium and/or tissue may be accelerated with increased pH level of the peptide hydrogel. In at least some non-limiting embodiments, self-assembly of peptides may begin after only about two seconds for peptide hydrogels at a pH level of between about 3.0 and about 3.5. In contrast, at a pH level of about 2.2, self-assembly may not begin until after about 10 to about 15 seconds. Without wishing to be bound by any particular theory, in accordance with one or more embodiments, faster gelation kinetics and/or rate of self-assembly may provide relatively better and/or longer contact between the peptide hydrogel and the mucosa beneficially allowing for improved healing and prevention of strictures.

In accordance with one or more embodiments, an administered peptide solution may have a pH level of about 3.5 or less. In some embodiments, the pH solution may have a pH level of about 3.0 to about 3.5. In some specific non-limiting examples, an administered peptide solution may have a pH level of about 3.4. Peptide hydrogels at an elevated pH level may exhibit a mechanical strength of at least about two to five times greater than that of the conventional peptide hydrogels in accordance with one or more embodiments. The tonicity of peptide solutions may be controlled using isotonic agents, including but not limited to salts such as sodium chloride, sugars such as sucrose and dextrose, and any mixture of isotonic agents such as phosphate-buffered saline (PBS). The specific conditions may also include adding a cation, such as a monovalent cation, to a self-assembling peptide solution.

The self-assembling peptide may be an amphiphilic self-assembling peptide. By “amphiphilic” it is meant that the peptide comprises hydrophobic portions and hydrophilic portions. In some embodiments, an amphiphilic peptide may comprise, consist essentially of, or consist of alternating hydrophobic amino acids and hydrophilic amino acids. By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid. The self-assembling peptide, also referred to herein as “peptide” may be administered to the pre-determined or desired target area in the form of a self-assembling peptide solution, composition, hydrogel, membrane, scaffold or other form. The hydrogel may also be referred to as a membrane or scaffold throughout this disclosure. The pre-determined or desired target area may be at or near the location of an ESD or EMR. The pre-determined or desired target area may be established based on the site of a polyp, tumor, such as a cancerous tumor, or other area that may have undergone a surgical procedure, or an unintentional or intentional trauma.

The self-assembling peptide solution may be an aqueous self-assembling peptide solution. The self-assembling peptide may be administered, applied, or injected in a solution that is substantially cell-free, or free of cells. In certain embodiments, the self-assembling peptide may be administered, applied, or injected in a solution that is cell-free or free of cells.

The self-assembling peptide solution may comprise, consist of, or consist essentially of the self-assembling peptide. The self-assembling peptide may be in a modified or unmodified form. By modified, it is meant that the self-assembling peptide may have one or more domains that comprise one or more amino acids that, when provided in solution by itself, would not self-assemble. By unmodified, it is meant that the self-assembling peptide may not have any other domains other than those that provide for self-assembly of the peptide. That is, an unmodified peptide consists of alternating hydrophobic and hydrophilic amino acids that may self-assemble into a beta-sheet, and a macroscopic structure, such as a hydrogel.

Administration of a solution may comprise, consist of, or consist essentially of administration of a solution having a pH level of 3.5 or less, the solution comprising, consisting of, or consisting essentially of a self-assembling peptide comprising, consisting of, or consisting essentially of between about 7 amino acids and about 32 amino acids. Other peptides that do not comprise, consist of, or consist essentially of between about 7 amino acids and about 32 amino acids may be contemplated by this disclosure.

By alternating, it is meant to include a series of three or more amino acids that alternate between a hydrophobic amino acid and a hydrophilic amino acid, and it need not include each and every amino acid in the peptide sequence alternating between a hydrophobic and a hydrophilic amino acid.

The methods may comprise administering a self-assembling peptide to a predetermined or desired target. The peptide may be administered as a hydrogel or form a hydrogel upon administration. A hydrogel is a term that may refer to a colloidal gel that is dispersed in water. The hydrogel may also be referred to as a membrane or scaffold throughout this disclosure. The methods may also comprise applying a self-assembling peptide to a predetermined or desired target as a solution such as an aqueous peptide solution.

The term “administering,” is intended to include, but is not limited to, applying, introducing or injecting the self-assembling peptide, in one or more of various forms including, but not limited to, by itself, by way of solution, such as an aqueous solution, or by way of a composition, hydrogel, or scaffold, with or without additional components.

In some embodiments, the target area is located within the esophagus, for example on an esophageal wound. The target area of the esophagus may be located about 5 cm above an esophagogastric junction. In some embodiments, the target area of the esophagus is located about 4 cm, about 5 cm, about 6 cm, about 7 cm, about 8 cm, about 9 cm, or about 10 cm above an esophagogastric junction. In some non-limiting embodiments, the target area of the esophagus may be between about 1 cm and about 10 cm in dimension. For example, the target area of the esophagus may be about 1 cm, about 2 cm, about 3 cm, about 4 cm, 5 cm in dimension, about 6 cm, about 7 cm, about 8 cm, about 9 cm, or about 10 cm in dimension.

The method may comprise introducing a delivery device at or near a predetermined or desired target area of a subject. The method may comprise introducing a delivery device comprising at least one of a syringe, pipette, tube, catheter, syringe catheter, or other needle-based device to the predetermined or desired target area of a subject. The self-assembling peptide may be administered by way of a syringe, pipette, tube, catheter, syringe catheter, or other needle-based device to the predetermined or desired target area of a subject. The gauge of the syringe needle may be selected to provide an adequate flow of a composition, a solution, a hydrogel, or a liquid from the syringe to the target area. This may be based in some embodiments on at least one of the amount of self-assembling peptide in a composition, peptide solution, or a hydrogel being administered, the concentration of the peptide solution, in the composition, or the hydrogel, and the viscosity of the peptide solution, composition, or hydrogel. The delivery device may be a conventional device or designed to accomplish at least one of to reach a specific target area, achieve a specific dosing regime, deliver a specific target volume, amount, or concentration, and deliver accurately to a target area.

The method of preventing or reducing esophageal stricture may comprise introducing a catheter into the subject and positioning an end of the catheter in a predetermined or target area, such as a portion of the gastrointestinal tract. The self-assembling peptide may be administered by way of a catheter to the target area in which at least a partial prevention or reduction in obstruction is desired. The use of a catheter may provide a more selective administration of the peptide to provide for a more accurate delivery to the target area. Selective administration of the peptide may allow for enhanced and more targeted delivery of the peptide solution, composition, or hydrogel such that prevention or reduction of esophageal stricture is successful and positioned in the desired location in an accurate manner. The selective administration may provide enhanced, targeted delivery that markedly improves the positioning and effectiveness of the treatment over use of a syringe or other delivery device. Delivery devices that may be used in the methods of the disclosure may include a syringe, pipette, tube, catheter, syringe catheter, other needle-based device, tube or catheter.

Use of the catheter may include use of accompanying devices, such as a guidewire used to guide the catheter into position, or an endoscope that may allow proper placement of the catheter and visualization of the target area, and/or the path to the target area. The endoscope may be a tube that may comprise at least one of a light and a camera or other visualization device to allow images of the subject's body to be viewed. The guidewire or endoscope may be introduced into the subject by way of the gastrointestinal tract. The endoscope may be introduced to the gastrointestinal tract prior to the introducing the catheter to the tract.

The use of the delivery device, such as a syringe, pipette, tube, catheter, syringe catheter, other needle-based device, catheter, or endoscope may require determining the diameter or size of the opening or tract in which there is a target area, such that at least a portion of the syringe, pipette, tube, syringe catheter, other needle-type device, catheter, or endoscope may enter the opening or tract to administer the peptide, peptide solution, composition, or hydrogel to the target area.

In certain embodiments, the hydrogel may be formed in vitro and administered to the desired location in vivo. In certain examples, this location may be the area in which it is desired to prevent or reduce stricture. In other examples, this location may be upstream, downstream of the area, or substantially near the area. It may be desired to allow a migration of the hydrogel to the area in which it is desired to prevent or reduce an obstruction or prevent or reduce stenosis. Alternatively, another procedure may position the hydrogel in the area in which it is desired. The desired location or target area may be at least a portion of an area in which tissue was removed, for example, in or around areas in which a cancerous or precancerous tissue was removed, in which one or more tumors was removed, or in which a biopsy was taken. The desired location or target area may be associated with an ESD or ERD procedure.

In certain aspects of the disclosure, the hydrogel may be formed in vivo. A solution comprising the self-assembling peptide, such as an aqueous solution, may be inserted to an in vivo location or area of a subject's esophagus to prevent esophageal stricture at that location. In certain examples, the hydrogel may be formed in vivo at one location, and allowed to migrate to the area in which it is desired to prevent or reduce an obstruction or prevent or reduce a stenosis. Alternatively, another procedure may place the hydrogel in the area in which it is desired to prevent stricture. The peptides of the present disclosure may be in the form of a powder, a solution, a gel, or the like. Since the self-assembling peptide gels in response to changes in solution pH and salt concentration, it can be distributed as a liquid that gels upon contact with a subject during application or administration.

In certain environments, the peptide solution may be a weak hydrogel and, as a result, it may be administered by way of a delivery device as described herein.

In accordance with one or more embodiments, a macroscopic scaffold is provided. The macroscopic scaffold may comprise, consist essentially of, or consist of a plurality of self-assembling peptides, each of which comprises, consists essentially of, or consists of between about 7 amino acids and about 32 amino acids in an effective amount that is capable of being positioned within a target area of a gastrointestinal tract, such as the esophagus, to promote healing and to prevent stricture.

In accordance with some embodiments, the self-assembling peptides may be amphiphilic, alternating between hydrophobic amino acids and hydrophilic amino acids. In accordance with one or more embodiments, a subject may be evaluated to determine a need for preventing esophageal stricture. Once the evaluation has been completed, a peptide solution to administer to the subject may be prepared.

In some embodiments, a biologically active agent may be used with the materials and methods of the present disclosure. A biologically active agent may comprise a compound, including a peptide, DNA sequence, chemical compound, or inorganic or organic compound that may impart some activity, regulation, modulation, or adjustment of a condition or other activity in a subject or in a laboratory setting. The biologically active agent may interact with another component to provide such activity. The biologically active agent may be referred to as a drug in accordance with some embodiments herein. In certain embodiments, one or more biologically active agents may be gradually released to the outside of the peptide system. For example, the one or more biologically active agents may be gradually released from the hydrogel. Both in vitro and in vivo testing has demonstrated this gradual release of a biologically active agent. The biologically active agent may be added to the peptide solution prior to administering to a subject, or may be administered separately from the solution to the subject.

This disclosure relates to aqueous solutions, hydrogels, scaffolds, and membranes comprising self-assembling peptides, sometimes referred to as self-assembling oligopeptides. The peptides may be comprised of a peptide having about 6 to about 200 amino acid residues. The self-assembling peptides may exhibit a beta-sheet structure in aqueous solution in the presence of physiological pH and/or a cation, such as a monovalent cation, or other conditions applicable to the gastrointestinal tract. The peptides may be amphiphilic and alternate between a hydrophobic amino acid and a hydrophilic amino acid. In certain embodiments, the peptide may comprise a first portion that may be amphiphilic, alternating between a hydrophobic amino acid and a hydrophilic amino acid, and another portion or region that is not amphiphilic.

The peptides may be generally stable in aqueous solutions and self-assemble into large, macroscopic structures, scaffolds, or matrices when exposed to physiological conditions, neutral pH, or physiological levels of salt. Once the hydrogel is formed it may not decompose, or may decompose or biodegrade after a period of time. The rate of decomposition may be based at least in part on at least one of the amino acid sequence and conditions of its surroundings.

By “macroscopic” it is meant as having dimensions large enough to be visible under magnification of 10-fold or less. In preferred embodiments, a macroscopic structure is visible to the naked eye. A macroscopic structure may be transparent and may be two-dimensional, or three-dimensional. Typically each dimension is at least 10 μm, in size. In certain embodiments, at least two dimensions are at least 100 μm, or at least 1000 μm in size. Frequently at least two dimensions are at least 1-10 mm in size, 10-100 mm in size, or more.

In certain embodiments, the size of the filaments may be about 10 nanometers (nm) to about 20 nm. The interfilament distance may be about 50 nm to about 80 nm.

“Physiological conditions” may occur in nature for a particular organism, cell system, or subject which may be in contrast to artificial laboratory conditions. The conditions may comprise one or more properties such as one or more particular properties or one or more ranges of properties. For example, the physiological conditions may include a temperature or range of temperatures, a pH or range of pH's, a pressure or range of pressures, and one or more concentrations of particular compounds, salts, and other components. For example, in some examples, the physiological conditions may include a temperature in a range of about 20 to about 40 degrees Celsius. In some examples, the atmospheric pressure may be about 1 atm. The pH may be in the range of a neutral pH. For example, the pH may be in a range of about 6 to about 8. The physiological conditions may include cations such as monovalent metal cations that may induce membrane or hydrogel formation. These may include sodium chloride (NaCl). The physiological conditions may also include a glucose concentration, sucrose concentration, or other sugar concentration, of between about 1 mM and about 20 mM. The physiological conditions may include the local conditions of the mouth, throat, esophagus, stomach, small intestine, large intestine, and rectum.

In some embodiments, the self-assembling peptides may be peptides of between about 6 amino acids and about 200 amino acids. In certain embodiments, the self-assembling peptides may be peptides of at least about 7 amino acids. In certain embodiments, the self-assembling peptides may be peptides of between about 7 amino acids and about 32 amino acids. In certain further embodiments, the self-assembling peptides may be peptides of between about 7 amino acids and about 17 amino acids. In certain other examples, the self-assembling peptides may be peptides of at least 8 amino acids, at least about 12 amino acids, or at least about 16 amino acids.

The peptides may also be complementary and structurally compatible. Complementary refers to the ability of the peptides to interact through ionized pairs and/or hydrogen bonds which form between their hydrophilic side-chains, and structurally compatible refers to the ability of complementary peptides to maintain a constant distance between their peptide backbones. Peptides having these properties participate in intermolecular interactions which result in the formation and stabilization of beta-sheets at the secondary structure level and interwoven filaments at the tertiary structure level.

Both homogeneous and heterogeneous mixtures of peptides characterized by the above-mentioned properties may form stable macroscopic membranes, filaments, and hydrogels. Peptides which are self-complementary and self-compatible may form membranes, filaments, and hydrogels in a homogeneous mixture. Heterogeneous peptides, including those which cannot form membranes, filaments, and hydrogels in homogeneous solutions, which are complementary and/or structurally compatible with each other may also self-assemble into macroscopic membranes, filaments, and hydrogels.

The membranes, filaments, and hydrogels may be non-cytotoxic. The hydrogels of the present disclosure may be digested and metabolized in a subject. The hydrogels may be biodegraded in about 30 days or more. They have a simple composition, are permeable, and are easy and relatively inexpensive to produce in large quantities. The membranes and filaments, hydrogels or scaffolds may also be produced and stored in a sterile condition. The optimal lengths for membrane formation may vary with at least one of the amino acid composition, solution conditions, and conditions at the target site.

In certain embodiments, a method of preventing esophageal stricture in a subject is provided. The method may comprise introducing a catheter into an esophagus of the subject. The method may further comprise administering through the catheter a solution having a pH level of about 3.5 or less, the solution comprising a self-assembling peptide comprising between about 7 amino acids and about 32 amino acids in an effective amount and in an effective concentration to form a hydrogel under esophageal conditions to provide prevention of esophageal stricture. The method may further comprise removing the catheter from the esophagus.

The method may further comprise visualizing a region or target area comprising at least a portion of the esophagus. Visualizing the region or target area may comprise visualizing the region or target area during at least one of identifying the target area of the gastrointestinal tract, introducing the catheter, positioning the end of the catheter in the target area, administering the solution, removing the catheter, and monitoring the gastrointestinal tract after removing the catheter. Visualizing the region or target area may provide for selective administration of the solution. Visualizing may occur at any time before, during, and after the administration of the solution. Visualization may occur, for example, at a time period of at least one of about one week subsequent to administration, about four weeks subsequent to administration and about eight weeks subsequent to administration. The method may further comprise monitoring the target area of the esophagus to determine an effectiveness of the administration of the solution. In some embodiments, monitoring the target area may include visualizing the target area. The target area may be monitored via endoscopy and/or radiology. The target area may be monitored at any day, for example within four weeks or eight weeks subsequent to administration. For instance, the target area may be monitored at days 3, 7, 14, 21, and 28 following administration of the solution. In some embodiments, visualizing and/or monitoring the target area may be performed to select an appropriate follow-on administration of peptide solution. For instance, visualizing and/or monitoring may be performed to determine whether a follow-on administration of peptide solution is desired.

The solution to be administered may consist essentially of, or consist of, a self-assembling peptide comprising at least about 7 amino acids. The solution to be administered may consist essentially of, or consist of, a self-assembling peptide comprising between about 7 amino acids and about 32 amino acids. The peptide may be amphiphilic and at least a portion of the peptide may alternate between a hydrophobic amino acid and a hydrophilic amino acid. The solution may have a pH level of about 3.5 or less, for example, about 3.4.

The amino acids of the self-assembling or amphiphilic peptides may be selected from d-amino acids, 1-amino acids, or combinations thereof. The hydrophobic amino acids may include Ala, Val, Ile, Met, Phe, Tyr, Trp, Ser, Thr and Gly. The hydrophilic amino acids may be basic amino acids, for example, Lys, Arg, His, Orn; acidic amino acids, for example, Glu, Asp; or amino acids which form hydrogen bonds, for example, Asn, Gln. Acidic and basic amino acids may be clustered on a peptide. The carboxyl and amino groups of the terminal residues may be protected or not protected. Membranes or hydrogels may be formed in a homogeneous mixture of self-complementary and self-compatible peptides or in a heterogeneous mixture of peptides which are complementary and structurally compatible to each other. Peptides fitting the above criteria may self-assemble into macroscopic membranes under suitable conditions, described herein.

The self-assembling peptides may be composed of about 6 to about 200 amino acid residues. In certain embodiments, about 7 to about 32 residues may be used in the self-assembling peptides, while in other embodiments self-assembling peptides may have about 7 to about 17 residues. The peptides may have a length of about 5 nm.

The peptides of the present disclosure may include peptides having the repeating sequence of arginine, alanine, aspartic acid and alanine (Arg-Ala-Asp-Ala (RADA)), and such peptide sequences may be represented by (RADA)ₚ, wherein p=2-50.

In accordance with one or more embodiments, the peptide hydrogel may be PuraMatrix® synthetic peptide hydrogel commercially available from 3-D Matrix Co., Ltd. In accordance with one or more other embodiments, the peptide hydrogel may be another self-assembling peptide such as KLDL12 having the sequence Lys-Leu-Asp-Leu-Lys-Leu-Asp-Leu-Lys-Leu-Asp-Leu (KLDL)₃. In accordance with one or more further embodiments, the peptide hydrogel may be IEIK13 having the sequence Ile-Glu-Ile-Lys-Ile-Glu-Ile-Lys-Ile-Glu-Ile-Lys-Ile (IEIK)₃I. Other peptide hydrogels exhibiting similar beneficial properties as discussed herein may also be used for similar indications. These peptide hydrogel solutions may be used at elevated pH levels as discussed herein.

Each of the peptide sequences disclosed herein may provide for peptides comprising, consisting essentially of, and consisting of the amino acid sequences recited. The present disclosure provides methods using hydrogels, and scaffolds comprising, consisting essentially of, or consisting of the peptides recited herein.

A 1 weight per volume (w/v) percent aqueous (water) solution and a 2.5 w/v percent of (RADA)₄ is available as the product PuraMatrix™ peptide hydrogel by 3-D Matrix Co., Ltd. PuraStat® synthetic peptide hydrogel solution (RADA16 2.5%) is also commercially available from 3-D Matrix Europe SAS.

The self-assembly of the peptides may be attributable to hydrogen bonding and hydrophobic bonding between the peptide molecules by the amino acids composing the peptides.

The self-assembling peptides of the present disclosure may have a nanofiber diameter in a range of about 10 nm to about 20 nm and an average pore size is in a range of about 5 nm to about 200 nm. In certain embodiments, the nanofiber diameter, the pore size, and the nanofiber density may be controlled by at least one of the concentration of peptide solution used and the amount of peptide solution used, such as the volume of peptide solution. As such, at least one of a specific concentration of peptide in solution and a specific amount of peptide solution to provide at least one of a desired nanofiber diameter, pore size, and density to adequately provide for an occlusion may be selected.

As used herein, an amount of a peptide, peptide solution or hydrogel effective to at least partially prevent or reduce esophageal stricture, an “effective amount” or a “therapeutically effective amount” refers to an amount of the peptide, peptide solution or hydrogel, which is effective, upon single or multiple administration (application or injection) to a subject, in treating, or in curing, alleviating, relieving or improving a subject with a disorder beyond that expected in the absence of such treatment. This may include a particular concentration or range of concentrations of peptide in the peptide solution or hydrogel and additionally, or in the alternative, a particular volume or range of volumes of the peptide solution or hydrogel.

The effective amount may be the amount required to achieve a prevention or reduction of esophageal stricture that is partial or complete. In some embodiments, a complete reduction of esophageal stricture is defined as achieving a lumen dimension that allows passage of a tubular device. For instance, a complete reduction of esophageal stricture may be defined as achieving a luminal dimension that allows passage of an endoscope. Partial reduction of esophageal stricture may comprise a reduction of about 50% of esophageal stricture, about 60% of esophageal stricture, about 70% of esophageal stricture, about 80% of esophageal stricture, or about 90% of esophageal stricture. In some embodiments, partial reduction of esophageal stricture includes any limitation in severity of the esophageal stricture.

In some embodiments, administration of the solution to the target area may maintain a predetermined lumen dimension at the target area of the esophagus. The predetermined lumen dimension may be associated with a diameter of the delivery device. For instance, in some embodiments, the predetermined lumen dimension may be at least about 10 mm, at least about 9 mm, or at least about about 8 mm. The self-assembling peptide may serve as a wound covering agent, providing both protection of the wound from acid aggression, and a scaffold for epithelial cell migration and wound healing.

The dosage, for example, volume or concentration, administered (for example, applied or injected) may vary depending upon the form of the peptide (for example, in a peptide solution, hydrogel, or in a dried form, such as a lyophilized form) and the route of administration utilized. The exact formulation, route of administration, volume, and concentration can be chosen in view of the subject's condition and in view of the particular target area or location that the peptide solution, hydrogel, or other form of peptide will be administered. Lower or higher doses than those recited herein may be used or required. Specific dosage and treatment regimens for any particular subject may depend upon a variety of factors, which may include the specific peptide or peptides employed, the dimension of the area that is being treated, the desired thickness of the resulting hydrogel that may be positioned in the desired target area, and the length of time of treatment. Other factors that may affect the specific dosage and treatment regimens include age, body weight, general health status, sex, time of administration, rate of degradation, the severity and course of the disease, condition or symptoms, and the judgment of the treating physician. In certain embodiments, the peptide solution may be administered in a single dose. In other embodiments, the peptide solution may be administered in more than one dose, or multiple doses. The peptide solution may be administered in at least two doses.

An effective amount and an effective concentration of the peptide solution may be selected to at least partially prevent or reduce esophageal stricture. In some embodiments, at least one of the effective amount and the effective concentration may be based in part on a dimension or diameter of the target area. In other embodiments, at least one of the effective amount and the effective concentration is based in part on the flow rate of one or more fluids at or near the target area. In still other embodiments, at least one of the effective amount and the effective concentration may be based in part on a dimension or diameter of a material being removed at the target site.

The effective amount may be, as described herein, an amount that may provide for an at least partial prevention or reduction in esophageal stricture. Various properties of the gastrointestinal tract may contribute to the selection or determination of the effective amount including at least one of the dimension or diameter of the target area, the flow rate of one or more fluids at or near the target area, the pH at or near the target area, and the concentration of various salts at or near the target area. One or more properties or conditions associated with the esophagus may at least partially influence or effect the selection or determination of the effective amount.

The effective amount may vary depending on the surface of the dissection. In some embodiments, the effective amount may include volumes of from about 0.1 milliliters (mL) to about 100 mL of a peptide solution. The effective amount may include volumes of from about 0.1 mL to about 10 mL of a peptide solution. In certain embodiments, the effective amount may be about 0.5 mL. In other embodiments, the effective amount may be about 1.0 mL. In yet other embodiments, the effective amount may be about 1.5 mL. In still yet other embodiments, the effective amount may be about 2.0 mL. In some other embodiments, the effective amount may be about 3.0 mL. In certain embodiments, the effective amount may be approximately 0.1 mL to about 5 mL per 1 cm² of target area. In certain embodiments, the effective amount may be approximately 1 mL per 1 cm² of target area. This effective amount may be used related to a concentration, such as a 2.5 weight per volume percent of a peptide solution of the present disclosure.

In some embodiments, a more effective prevention or reduction in stricture may be achieved with a greater volume of peptide solution administered or a higher concentration of peptide in solution to be administered. This may allow a longer or thicker hydrogel to form within the target area, allowing a more secure position of the hydrogel in the target area. It is possible that if a high enough volume is not selected, the hydrogel may not be effective in preventing or reducing a gastrointestinal obstruction in the target area for the desired period of time.

The effective concentration may be, as described herein, an amount that may provide for a desired prevention or reduction in esophageal stricture. Various properties of the gastrointestinal tract may contribute to the selection or determination of the effective concentration including at least one of a dimension or diameter of the target area, the flow rate of one or more fluids at or near the target area, and on a dimension or diameter of a material being removed from the target site.

The effective concentration may include peptide concentrations in the solution in a range of about 0.1 weight per volume (w/v) percent to about 10 w/v percent. The effective concentration may include peptide concentrations in the solution in a range of about 0.1 w/v percent to about 3.5 w/v percent. In certain embodiments, the effective concentration may be about 1 w/v percent. In other embodiments, the effective concentration may be about 2.5 w/v percent. In yet other embodiments, the effective concentration may be about 3.0 w/v percent.

In certain embodiments, a peptide solution having a higher concentration of peptide may provide for a more effective hydrogel that has the ability to stay in place and provide effective prevention or reduction in esophageal stricture. For purposes of delivering the peptide solution, higher concentrations of peptide solutions may become too viscous to allow for effective and selective administration of the solution. It is possible that if a high enough concentration is not selected, the hydrogel may not be effective in maintaining a prevention or reduction in stricture at the target area for the desired period of time. The effective concentration may be selected to provide for a solution that may be administered by injection or other means using a particular diameter or gauge catheter or needle.

Methods of the disclosure contemplate single as well as multiple administrations of a therapeutically effective amount of the peptides, compositions, peptide solutions, membranes, filaments, and hydrogels as described herein. Peptides as described herein may be administered at regular intervals, depending on the nature, severity and extent of the subject's condition. In some embodiments, a peptide, composition, peptide solution, membrane, filament, or hydrogel may be administered in a single administration. In some embodiments, a peptide, composition, peptide solution, or hydrogel described herein is administered in multiple administrations. In some embodiments, a therapeutically effective amount of a peptide, composition, peptide solution, membrane, filament, or hydrogel may be administered periodically at regular intervals. The regular intervals selected may be based on any one or more of the initial peptide concentration of the solution administered, the amount administered, and the degradation rate of the hydrogel formed. For example, after an initial administration, a follow-on administration may occur after, for example, one week, two weeks, four weeks, six weeks, or eight weeks. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject. The pre-determined intervals may be the same for each follow-on administration, or they may be different. In some embodiments, a peptide, peptide solution, or hydrogel may be administered chronically at pre-determined intervals to maintain at least a partial prevention or reduction in gastrointestinal obstruction in a subject over the life of the subject. The pre-determined intervals may be the same for each follow-on administration, or they may be different. This may be dependent on whether the hydrogel formed from the previous administration is partially or totally disrupted or degraded. The follow-on administration may comprise administration of a solution having the same concentration of peptide and volume as the initial administration, or may comprise administration of a solution of lesser or great concentration of peptide and volume. The selection of the appropriate follow-on administration of peptide solution may be based on imaging the target area and the area surrounding the target area and ascertaining the needs based on the condition of the subject.

The self-assembling peptides of the present disclosure, such as RADA16, may be peptide sequences that lack a distinct physiologically or biologically active motif or sequence, and therefore may not impair intrinsic cell function. Physiologically active motifs may control numerous intracellular phenomena such as transcription, and the presence of physiologically active motifs may lead to phosphorylation of intracytoplasmic or cell surface proteins by enzymes that recognize the motifs. When a physiologically active motif is present in a peptide tissue occluding agent, transcription of proteins with various functions may be activated or suppressed. The self-assembling peptides, of the present disclosure may lack such physiologically active motifs and therefore do not carry this risk.

A sugar may be added to the self-assembling peptide solution to improve the osmotic pressure of the solution from hypotonicity to isotonicity without reducing the tissue occluding effect, thereby allowing the biological safety to be increased. In certain examples, the sugar may be sucrose or glucose.

The optimal lengths for membrane formation may vary with the amino acid composition. A stabilization factor contemplated by the peptides of the present disclosure is that complementary peptides maintain a constant distance between the peptide backbones. Peptides which can maintain a constant distance upon pairing are referred to herein as structurally compatible. The interpeptide distance can be calculated for each ionized or hydrogen bonding pair by taking the sum of the number of unbranched atoms on the side-chains of each amino acid in the pair.

The initial concentration of the peptide may be a factor in the size and thickness of the membrane, hydrogel, or scaffold formed. In general, the higher the peptide concentration, the higher the extent of membrane or hydrogel formation. Hydrogels, or scaffolds formed at higher initial peptide concentrations (about 10 mg/ml) (about 1.0 w/v percent) may be thicker and thus, likely to be stronger.

Formation of the, membranes, hydrogels, or scaffolds may be very fast, on the order of a few minutes. The formation of the membranes or hydrogels may be irreversible. In certain embodiments, the formation may be reversible, and in other embodiments, the formation may be irreversible. The hydrogel may form instantaneously upon administration to a target area. The formation of the hydrogel may occur within about one to two minutes of administration. In other examples, the formation of the hydrogel may occur within about three to four minutes of administration. In certain embodiments the time it takes to form the hydrogel may be based at least in part on one or more of the concentration of the peptide solution, the volume of peptide solution applied, and the conditions at the area of application or injection (for example, the concentration of monovalent metal cations at the area of application, the pH of the area, and the presence of one or more fluids at or near the area). The process may be unaffected by pH of less than or equal to 12, and by temperature. The membranes or hydrogels may form at temperatures in the range of about 1 to 99 degrees Celsius.

The hydrogels may remain in position at the target area for a period of time sufficient to provide a desired effect using the methods of the present disclosure. The desired effect may be to at least partially prevent or reduce esophageal stricture associated with a surgical procedure such as endoscopic mucosal resection (EMR) or endoscopic submucosal dissection (ESD).

The period of time that the membranes or hydrogels may remain at the desired area may be for one or more days, up to one or more weeks. In other examples, it may remain at the desired area for up to 30 days, or more. It may remain at the desired area indefinitely. In other examples, it may remain at the desired area for a longer period of time, until it is naturally degraded or intentionally removed. If the hydrogel naturally degrades over a period of time, subsequent application or injection of the hydrogel to the same or different location may be performed.

In certain embodiments, the self-assembling peptide may be prepared with one or more components that may provide for enhanced effectiveness of the self-assembling peptide or may provide another action, treatment, therapy, or otherwise interact with one or more components of the subject. For example, additional peptides comprising one or more biologically or physiologically active amino acid sequences or motifs may be included as one of the components along with the self-assembling peptide. Other components may include biologically active compounds such as a drug or other treatment that may provide some benefit to the subject. For example, a cancer treating drug or anticancer drug may be administered with the self-assembling peptide, or may be administered separately.

The peptide, peptide solution, or hydrogel may comprise small molecular drugs to treat the subject or to prevent hemolysis, inflammation, and infection. The small molecular drugs may be selected from the group consisting of glucose, saccharose, purified saccharose, lactose, maltose, trehalose, destran, iodine, lysozyme chloride, dimethylisoprpylazulene, tretinoin tocoferil, povidone iodine, alprostadil alfadex, anise alcohol, isoamyl salicylate, α,α-dimethylphenylethyl alcohol, bacdanol, helional, sulfazin silver, bucladesine sodium, alprostadil alfadex, gentamycin sulfate, tetracycline hydrochloride, sodium fusidate, mupirocin calcium hydrate and isoamyl benzoate. Other small molecular drugs may be contemplated. Protein-based drugs may be included as a component to be administered, and may include erythropoietin, tissue type plasminogen activator, synthetic hemoglobin and insulin.

A component may be included to protect the peptide solution against rapid or immediate formation into a hydrogel. This may include an encapsulated delivery system that may degrade over time to allow a controlled time release of the peptide solution into the target area to form the hydrogel over a desired, predetermined period of time. Biodegradable, biocompatible polymers may be used, such as ethylene vinyl acetate, polyanhydrides, polyglycolic acid, collagen, polyorthoesters, and polylactic acid.

In accordance with one or more embodiments, the peptide solution may include one or more anti-inflammatory molecules such as triamcinolone, and/or wound healing stimulants such as epidermal growth factor, in order to limit fibrogenesis while enhancing epithelial growth.

Any of the components described herein may be included in the peptide solution or may be administered separate from the peptide solution. Additionally, any of the methods and methods of facilitating provided herein may be performed by one or more parties.

In accordance with one or more embodiments, a self-assembling peptide solution may form a scaffold that acts as a wound dressing, opposing mechanical aggression while closely mimicking the structure and porosity of extracellular matrices, as well as promotes epithelial growth, and/or reduces inflammatory and fibrogenic activity in the esophagus.

The function and advantage of these and other embodiments of the methods disclosed herein will be more fully understood from the example below. The following example is intended to illustrate the benefits of the disclosed treatment approach, but does not exemplify the full scope thereof.

### Example

A randomized, controlled trial was conducted in a porcine model to assess the use of a self-assembling peptide (SAP) matrix (PuraStat® 2.5% synthetic peptide hydrogel, pH 3.4, 3-D Matrix Europe SAS, Caluire-et-Cuire, France) for the prevention of esophageal stricture after endoscopic resection.

### Preliminary Experiments

Preliminary experiments were conducted to assess six modalities of PuraStat® synthetic peptide hydrogel application in the esophagus: submucosal injection of the PuraStat® at pH 2.2 before ESD (one pig); injection of the PuraStat® at pH 2.2 in the residual submucosa (one pig); standard application of PuraStat® at pH 2.2 on the esophageal wound after the procedure, using a dedicated 2.8 mm catheter (2 pigs); standard application of PuraStat® at pH 3.1 on the esophageal wound after the procedure, using a dedicated 2.8 mm catheter (2 pigs); standard application of PuraStat® at pH 3.4 on the esophageal wound after the procedure, using a dedicated 2.8 mm catheter (2 pigs); application of PuraStat® at pH 2.2 followed by spraying of hypertonic saline (2 pigs), in order to haste the peptide matrix assembly. Only one pig in the standard application group with PuraStat® pH 3.4 had a mild esophageal stricture at day 14, whereas all others had tight strictures. Furthermore, the persistence of PuraStat® adherent to the esophageal wounds was observed at day 1 and 2 after application.

Based on these data, it was decided to use the standard application protocol with 6 mL of PuraStat® pH 3.4 applied with a dedicated catheter immediately after the CESD.

### Experimental Design

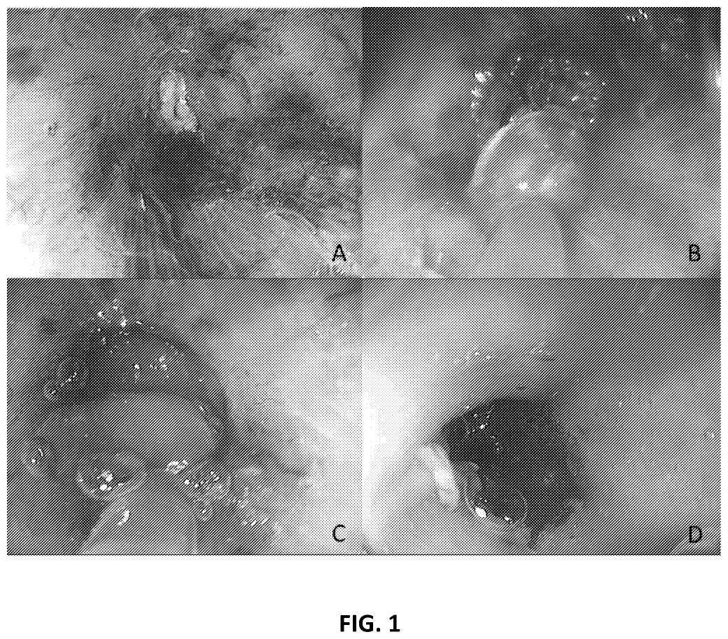

Ten pigs were randomly assigned to the SAP or the control group after undergoing a 5 cm long circumferential endoscopic submucosal dissection of the lower esophagus, 5 cm above the esophagogastric junction. Pictures illustrating the technique used for application of the SAP to the target area are presented in FIGS. 1A-1D:

A: Deep submucosa and muscularis propria exposed after circumferential endoscopic submucosal dissection.

B: First drop of SAP sticking to the esophageal wall.

C: Further application of the SAP

D: Final aspect upon removal of the endoscope, with the esophagus filled with SAP.

Post-operative care was as follows: the animals were daily assessed by animal caretakers, weekly by a veterinarian. A liquid diet was resumed on the first postoperative day for three days, followed by semi-liquid feeding. Antibiotherapy with amoxicillin and clavulanate 1 g×2/day was given for the first three post-operative days. Finally, esomeprazole 40 mg/d was administered during all the study period after ESD.

When a symptomatic esophageal stricture, defined by an inability to reach the stomach with a 9 mm endoscope and associated symptoms (anorexia, regurgitations, weight loss) was diagnosed, animals were euthanized. All the animals were euthanized after 28 days from CESD, and underwent necropsy with en bloc esophagectomy.

Esophageal diameter on endoscopy and esophagogram, weight variation, and histological measurements of fibrosis, granulation tissue, and neoepithelium were assessed in each animal. Clinical data collected on a daily basis included behaviour, a quantitative and qualitative assessment of food intake and occurrence of regurgitations or vomiting, whereas animals were weighed once per week. The esophageal width was measured every week at the level of the mid-oesophagus and the upper limit of CESD during sedated upper endoscopy. Furthermore, an esophagogram was performed at day 14 and in case of symptoms suspicious of an esophageal stricture.

### Results

General clinical condition and animal behavior remained normal throughout the study, despite major weight loss and regurgitations in some animals. At day 7, all animals had a normal food tolerance and no esophageal stricture had occurred.

At day 14 after CESD, 40% (2/5) of pigs in the SAP treated group and 100% (5/5) in the control group (p=0.04) had developed symptomatic esophageal stricture. Median (IQR) esophageal diameter was 8 (2.5-9) mm in the SAP group vs. 4 (3-4) in the control group (p=0.13). The median (IQR) stricture indexes at day 14 were 0.32 (0.14-0.48) and 0.26 (0.14-0.33) in treated and control groups, respectively (p=0.42). Main clinical and endoscopic outcome measurements are presented in Table 1.

TABLE 1
Weight loss, esophageal diameter variation in pigs treated with
self-assembling peptide and controls at day 14.

| | Treated with SAP | Controls | |
|---|---|---|---|
| | (n = 5) | (n = 5) | p |
| Weight variation (kg) - | 0.2 (−7.4-1.8) | −3.8 (−5.4-0.6) | 0.9 |
| median (IQR) | | | |
| Esophageal diameter - | 8 (2.5-9) | 4 (3-4) | 0.13 |
| median (IQR) | | | |
| Stricture index - median | 0.32 (0.14-0.48) | 0.26 (0.14-0.33) | 0.42 |
| (IQR) | | | |
| Symptomatic stricture | 40% | 100% | 0.04 |
| rate (%) | | | |

*: between day 0 and day 14
SAP: self-assembling peptide
IQR: interquartile range

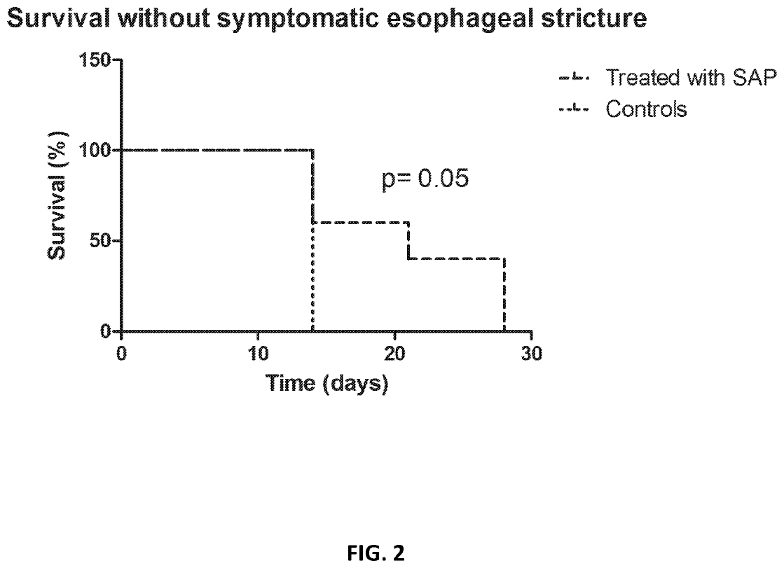

Three animals of the group treated with SAP ultimately developed symptomatic esophageal strictures at day 21 and 28, as shown in FIG. 2 (survival without symptomatic esophageal stricture).

Median (IQR) weight variation during the study were +0.2 (−7.4; +1.8) and −3.8 (−5.4; +0.6) in the treated and control groups, respectively (p=0.9). Pathology results are presented in Table 2. There were no statistically significant differences between groups in terms of esophageal inflammation, fibrosis, or epithelial growth.

TABLE 2
Pathology outcomes in pigs treated with
self-assembling peptide and controls.

| | Treated with SAP | | Controls | | |
|---|---|---|---|---|---|
| | (n = 5) | | (n = 5) | | p |
| Granulation tissue* - | 118 | (101-177) | 122 | (97-187) | 0.9 |
| median (IQR) | | | | | |
| Type of inflammatory | | | | | |
| cell infiltrate | | | | | |
| Acute | 20% | (1/5) | 80% | (4/5) | 0.2 |
| Chronic | 80% | (4/5) | 20% | (1/5) | |
| Esophageal fibrosis* - | 262 | (144-341) | 170 | (129-210) | 0.2 |
| median (IQR) | | | | | |
| Neo-epithelium* - | 630 | (269-3513) | 1155 | (706-1778) | 0.7 |
| median (IQR) | | | | | |

SAP: self-assembling peptide
IQR: interquartile range
*in μm

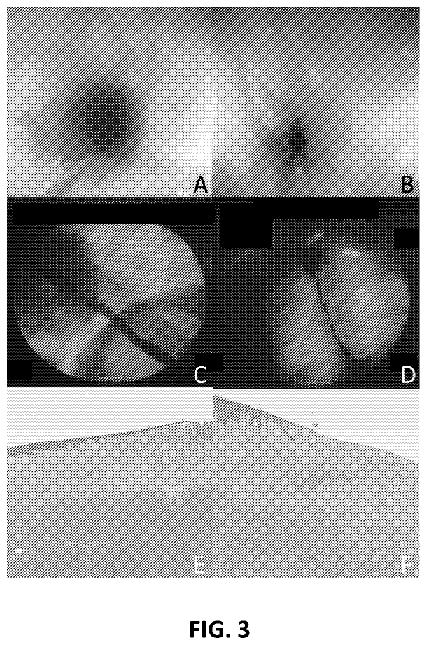

Representative endoscopic, radiological, and pathology findings of each group at day 14 are shown in FIGS. 3A-3F:

A: Endoscopic view in a pig treated with SAP, showing a moderate narrowing of the esophagus and smooth, reepithelialised esophageal wall.

B: Endoscopic view in a control pig, showing a severe stricture with granulation tissue and persistent inflammation.

C: Barium esophagogram in the same pig treated with SAP, showing a mild narrowing of the esophagus.

D: Barium esophagogram in the same control pig, showing a severe narrowing of the esophagus.

E and F: Representative histological view in a pig treated with SAP (E) and a control (F), showing the neoepithelium overgrowing the inflammatory infiltrate and fibrotic layer (Hematoxylin Eosin and Saffron, 25×).

The application of the SAP matrix (PuraStat® 2.5% synthetic peptide hydrogel, pH 3.4) on esophageal wounds after circumferential endoscopic submucosal dissection decreased the onset of esophageal stricture. The rate of esophageal stricture at day 14 was 40% in the SAP treated group vs. 100% in the control group (p=0.04). Median (IQR) esophageal diameter at day 14 was 8 mm (2.5-9) in the SAP group vs. 4 mm (3-4) in the control group (p=0.13). The median (IQR) stricture indexes on esophagograms at day 14 were 0.32 (0.14-0.48) and 0.26 (0.14-0.33) in treated and control groups, respectively (p=0.42). Median (IQR) weight variation during the study was +0.2 (−7.4; +1.8) and −3.8 (−5.4; +0.6) in the treated and control groups, respectively (p=0.9). Fibrosis, granulation tissue, and neoepithelium were not significantly different between the groups.

### CONCLUSIONS

PuraStat® SAP matrix (pH 3.4) was demonstrated to be a useful wound dressing for esophageal ulcers following circumferential endoscopic resection of the esophagus. It significantly prevented the occurrence of esophageal stricture in this study. It was demonstrated that PuraStat® SAP matrix (pH 3.4) significantly reduced the rate of esophageal stricture after circumferential esophageal ESD, and was associated with a doubled esophageal diameter at day 14 after CESD. The effect on stricture prevention was not sustainable, since all animals eventually developed esophageal strictures by day 28. Thus, the SAP matrix most likely delayed the onset of stricture formation, suggesting that the fibrogenic process starts over again as soon as the SAP has been washed away or degraded. Additional administrations of the SAP matrix could address this effect.

### Kinetics of Peptide Hydrogel Detection

PuraStat® is applied to an esophageal wound after procedure, following standard methods. The site is monitored at days 7, 10, and 14 after application, for presence of PuraStat®. The site may be monitored visually, using an endoscope, microscopically, and/or using mass spectrometry. It is expected that at least a microscopic quantity of PuraStat® will be detected each of the days monitored.

Encapsulation of Corticosteroids into Peptide Hydrogel

The current reference treatment for prevention of esophageal stenosis after endoscopic treatment is corticosteroid therapy, administered orally or applied to the esophagus by endoscopic injections. The effectiveness of oral and applied corticosteroid therapy is suboptimal. The mode of application of corticosteroid by injection into the esophageal wall is risky. A study may be performed to evaluate the effectiveness of endoscopic apposition of a matrix of self-assembled peptides on the esophageal excision area, and its combination with a local corticosteroid.

ESD is performed on 35 animal models. Of the 35 animals, 5 are treated with the ESD procedure alone, 15 receive an application of PuraStat® on the esophageal wound site, following standard methods, and 15 receive an application of PuraStat® with triamcinolone on the esophageal wound site, following standard methods. The effect of the different treatments will be evaluated by the rate of esophageal stenosis at 14 days and 28 days after application of sample.

It is expected that the animals treated with the PuraStat® with triamcinolone will show an absence or decrease of esophageal stenosis, verified by endoscopic examination and documented by histological and biological analysis.

What is claimed is:

1. A method of preventing esophageal stricture following an endoscopic resection procedure in a subject, comprising:
introducing a delivery device to an esophagus of the subject;
positioning an end of the delivery device at a target area of the esophagus where prevention of esophageal stricture is desired;
administering through the delivery device a solution having a pH level of about 3.5 or less, the solution including a self-assembling peptide comprising between about 7 amino acids and about 32 amino acids in an effective amount and in an effective concentration to form a hydrogel under esophageal conditions to provide prevention of esophageal stricture; and
removing the delivery device from the esophagus.

2. The method of claim 1, wherein the endoscopic resection procedure relates to superficial neoplasm of the esophagus.

3. The method of claim 2, wherein the endoscopic resection procedure is either circumferential endoscopic submucosal dissection or circumferential endoscopic mucosal resection.

4. The method of claim 1, wherein administering the solution provides prevention of inflammatory and/or fibrotic patterns.

5. The method of claim 4, wherein administering the solution speeds reepithelialization and/or inhibits acid aggression at the target area of the esophagus.

6. The method of claim 5, wherein administering the solution provides a scaffold for epithelial cell migration and/or healing at the target area of the esophagus.

7. The method of claim 1, wherein the target area of the esophagus is located about 5 cm above an esophagogastric junction.

8. (canceled)

9. The method of claim 1, wherein the target area of the esophagus is between about 1 cm and about 10 cm in dimension.

10. The method of claim 1, wherein the effective amount is approximately 1 mL per 1 cm² of target area.

11. The method of claim 1, wherein the concentration effective to provide prevention of esophageal stricture comprises a concentration in a range of about 0.1 weight per volume (w/v) percent to about 3 w/v percent peptide.

12. The method of claim 1, wherein the amount effective to provide prevention of esophageal stricture comprises a volume in a range of about 0.1 mL to about 10 mL.

13. The method of claim 1, further comprising administering a corticosteroid at the target area of the esophagus.

14-19. (canceled)

20. The method of claim 1, wherein the solution consists essentially of an amphiphilic peptide comprising at least 12 amino acids that alternate between a hydrophobic amino acid and a hydrophilic amino acid.

21. (canceled)

22. (canceled)

23. The method of claim 1, wherein the subject is human.

24-26. (canceled)

27. The method of claim 1, wherein the peptide in the solution comprises RADA16 (SEQ ID NO: 7) or IEIK13 (SEQ ID NO: 6).

28. (canceled)

29. The method of claim 1, wherein administration of the solution maintains a predetermined lumen dimension at the target area of the esophagus and the predetermined lumen dimension is at least about 10 mm.

30. (canceled)

31. The method of claim 1, wherein the solution is buffered with an alkali salt selected from sodium hydroxide, sodium chloride, potassium hydroxide, calcium hydroxide, sodium carbonate, sodium acetate, and sodium sulfide.

32. (canceled)

33. The method of claim 1, wherein the pH level of the solution is about 3.4.

34. (canceled)

35. (canceled)

36. The method of claim 1, wherein the solution further comprises an anti-inflammatory molecule and/or a wound healing stimulant.

37. (canceled)

38. (canceled)

39. The method of claim 36, wherein the anti-inflammatory molecule is triamcinolone and the wound healing stimulant is epidermal growth factor.
